(19) United States
(12) Patent Application Publication
(10) Pub. No.: US 2024/0165297 A1
(43) Pub. Date: May 23, 2024

(54) HYDROGEL DELIVERY MATRIX

(71) Applicant: Philip Neil MacPherson, Auckland (NZ)

(72) Inventor: Philip Neil MacPherson, Auckland (NZ)

(73) Assignee: Philip Neil MacPherson, Auckland (NZ)

(21) Appl. No.: 18/235,404

(22) Filed: Aug. 18, 2023

Related U.S. Application Data
(60) Provisional application No. 63/399,456, filed on Aug. 19, 2022.

(51) Int. Cl. A61L 26/00

(52) U.S. Cl. CPC A61L 26/0057; A61L 26/0023; A61L 26/0066; A61L 26/008; A61L 2300/252; A61L 2300/404; A61L 2300/406; A61L 2300/41; A61L 2300/412; A61L 2430/02; A61L 2430/12; A61L 2430/38

(57) ABSTRACT

The present disclosure relates to delivering a biological material to a wounded tissue of a patient by the application of a composition comprising biocompatible modified starch particles modified by carboxylation or hydroxylation of glucose units in the starch. Compositions comprising biocompatible modified starch particles are also provided.

### TECHNICAL FIELD

The disclosure relates to delivering a biological material to a wounded tissue of a patient by the application of a composition comprising biocompatible modified starch particles modified by carboxylation or hydroxylation of glucose units in the starch. Compositions comprising biocompatible modified starch particles are also provided.

### BACKGROUND

Many types of injury can result in wounds where tissue must be removed. For example, damaged or diseased tissue may need to be removed to promote healing. In such cases, it may not be sufficient to merely close the wound, but may also be necessary to provide a tissue graft.

Biological material such as tissue fragments may be incorporated into a wound to aid healing, for example as a tissue graft. The need for tissue grafting has been appreciated throughout human history (Hernigou 2014), and bone grafts have been used since at least 1668, when bone from a dog's calvarium was successfully grafted to a soldier's skull (Older 1992).

Autogenous bone grafts are considered the “gold standard” for bone repair (Schmidt 2021). An ideal bone graft material requires several properties: it must be biocompatible and bioresorbable so that it doesn't impair the natural healing process; it should ideally provide some level of structural support, although this can be supplemented with extrinsic supports such as pins, rods, casts, etc.; it should also support osteogenesis, osteoinduction, and osteoconduction. Osteogenesis refers to the ability of the bone graft to provide bone-producing cells, such as osteoblasts; osteoinduction refers to its ability to release factors (such as growth factors) that promote the recruitment, proliferation, and differentiation of stem cells; and osteoconduction refers to its ability to provide a physical support scaffold upon which bone formation can occur. Autogenous bone grafts meet all these criteria.

Autogenous bone grafts require harvesting bone material from the patient, usually from a distal site such as the iliac crest. This can present problems, however, as it usually requires a separate surgical incision, and can result in donor site morbidity and pain (Younger and Chapman 1989).

Autogenous bone graft material can also be harvested from the material removed during surgery that would otherwise be discarded. For example, spinal fusion surgery is sometimes used to treat deformities of the spine, spinal weakness or instability, herniated disks, or chronic back pain. This surgery involves permanently fusing two or more vertebrae, eliminating motion between then, by removing some bone material and placing bone graft material between the vertebrae to encourage bone growth and fusion. “Bone dust” created by the removal of bone during the surgery can be used as a source of the bone graft material (Gao et al. 2018; Street et al. 2017). Bone dust can release osteogenic factors that encourage the proliferation of osteoblasts, and can be a source of osteogenic cells.

However, the amount of bone dust harvested during such surgeries may be insufficient. Additionally, the bone dust may not be of the ideal malleability, viscosity, or adhesiveness for application to the wounded tissue. For example, the bone dust may not stay in place, leading to undesirable surgical outcomes. This can include heterotopic ossification, in which extraskeletal bone forms in muscle and soft tissues, usually as a result of trauma or surgery (Meyers et al. 2019).

Accordingly, it is desirable to provide methods for delivering a biological material to a wounded tissue of a patient by administering compositions. These compositions should be easy and safe to handle, and have sufficient malleability, viscosity, cohesiveness, and adhesiveness to be easy to handle during application, conform to the wound defect, and remain in place during wound closure. The compositions should be biocompatible, bioabsorbable, and should allow and support the migration of cells into the wound defect. The compositions may also contain additional factors to improve wound healing, such as growth factors, antimicrobials, and/or anti-inflammatory factors.

The present disclosure addresses these desiderata, or at least provides the public with a useful choice.

### SUMMARY OF THE INVENTION

In a first aspect, the disclosure provides a method of delivering a biological material to a wounded tissue of a patient, the method comprising:
a. combining:
   i. a composition comprising biocompatible modified starch particles, wherein the starch particles are modified by carboxylation or hydroxylation of glucose units in the starch, and
   ii. the biological material to form a mixture, and
b. applying the mixture to the wounded tissue of the patient.

In a second aspect, the disclosure provides a method of delivering a biological material to a wounded tissue of a patient, the method comprising:
applying a mixture to the wounded tissue of the patient, the mixture having been obtained by combining to form a mixture,
   a. a composition comprising biocompatible modified starch particles, wherein the starch particles are modified by carboxylation or hydroxylation of glucose units in the starch, and
   b. the biological material.

In one embodiment the biological material comprises a biological fluid. In one embodiment the biological fluid comprises blood, blood plasma, and/or lymph.

In one embodiment the biological material comprises one or more tissue fragments. In one embodiment the one or more tissue fragments comprise autogenous tissue fragments from the patient. In one embodiment the one or more tissue fragments comprise allogenic tissue fragments from a donor. In one embodiment the tissue fragments comprise bone dust or cartilage.

In one embodiment the mixture comprises growth factors, cytokines, anabolic factors, osteoinductive factors, and/or viable cells.

In one embodiment the mixture has a viscosity at 25° C. of at least 50 mPa·s, preferably a viscosity of from 30,000 to 10⁶ mPa·s.

In one embodiment the biological material is combined with the biocompatible modified starch particles in a ratio of from about 5:1 to about 20:1, preferably in a ratio of about 10:1.

In one embodiment the composition comprising biocompatible modified starch particles is in the form of a powder.

In one embodiment the composition comprising biocompatible modified starch particles is in the form of a gel, putty, or paste.

In one embodiment the method comprises delivering a biological material in lumbar fusion surgery, maxillofacial surgery, oral surgery, dental surgery, or cranial surgery. In one embodiment the biological material is harvested from material removed during the surgery.

In one embodiment the combining comprises combining:
a. a composition comprising biocompatible modified starch particles, wherein the starch particles are modified by carboxylation or hydroxylation of glucose units in the starch,
b. the biological material, and
c. a liquid.

In one embodiment the liquid comprises sterile saline solution and/or a biological fluid from the patient. In one embodiment the ratio of biocompatible modified starch particles to liquid is from 1:1 to 1:30, preferably from 1:5 to 1:15. In one embodiment the liquid comprises one or more bioactive agents selected from osteogenic, anti-inflammatory, antimicrobial, antibacterial, and/or antibiotic agents.

Additionally or alternatively, the liquid may comprise one or more therapeutic agents. Examples of such agents include, but are not limited to, pharmaceutical agents, growth factors, cytokines, anabolic factors, osteoinductive factors, bone morphogenic proteins (BMPs), tissue growth enhancing agents, and/or viable cells.

In one embodiment the composition comprises one or more bioactive agents selected from osteogenic, anti-inflammatory, antimicrobial, antibacterial, and/or antibiotic agents. Additionally or alternatively, the composition may comprise one or more therapeutic agents, as defined above.

In one embodiment the combining comprises combining:
a. a composition comprising biocompatible modified starch particles, wherein the starch particles are modified by carboxylation or hydroxylation of glucose units in the starch,
b. the biological material, and
c. one or more bioactive agents selected from osteogenic, anti-inflammatory, antimicrobial, antibacterial, and/or antibiotic agents.

Optionally, the combining further comprises combining d) one or more therapeutic agents. Examples of such agents include, but are not limited to, pharmaceutical agents, growth factors, cytokines, anabolic factors, osteoinductive factors, bone morphogenic proteins (BMPs), tissue growth enhancing agents, and/or viable cells.

In one embodiment the one or more bioactive agents comprise lactoferrin.

In one embodiment the composition is sterile. In one embodiment the composition has a biologically acceptable level of heavy metal contamination.

In one embodiment the biocompatible modified starch particles are degradable by amylase. In one embodiment the biocompatible modified starch particles are carboxymethyl starch particles. In one embodiment the biocompatible modified starch particles are cross-linked. In one embodiment the biocompatible modified starch particles are pre-gelatinised modified starch particles. In one embodiment the biocompatible modified starch particles are non-porous. In one embodiment the biocompatible modified starch particles are substantially devoid of a microporous surface.

In a third aspect, the disclosure provides a composition comprising:
a. biocompatible modified starch particles;
b. optionally a liquid; and
c. a biological material;
wherein the composition is malleable and cohesive.

In a fourth aspect, the disclosure provides a composition comprising:
a. biocompatible modified starch particles;
b. optionally a liquid; and
c. a biological material;
wherein the weight ratio of biocompatible modified starch particles to saline and/or biological material is from 1:1 to 1:100, preferably from 1:5 to 1:15.

In one embodiment the liquid comprises saline.

In one embodiment the composition has a viscosity at 25° C. of at least 50 mPa·s, preferably a viscosity of from 30,000 to 10⁶ mPa·s.

In one embodiment the composition is for use in surgery. In one embodiment the composition is for use in a method according to the first or second aspects.

In a fifth aspect, the disclosure provides a composition comprising biocompatible modified starch particles for use in a method of delivering a biological material to a wounded tissue of a patient, wherein the biocompatible modified starch particles are modified by carboxylation or hydroxylation of glucose units in the starch, and wherein the method comprises applying the composition to the wounded tissue of the patient, and wherein the method comprises combining the composition with the biological material before or during the application to the wounded tissue.

In a sixth aspect, the disclosure provides use of biocompatible modified starch particles in the manufacture of a medicament for delivering a biological material to a wounded tissue of a patient, wherein the biocompatible modified starch particles are modified by carboxylation or hydroxylation of glucose units in the starch.

In a seventh aspect, the disclosure provides a non-surgical method for preparing a mixture, comprising combining:
a. a composition comprising biocompatible modified starch particles, wherein the starch particles are modified by carboxylation or hydroxylation of glucose units in the starch,
b. a biological material, and
c. optionally saline,
thus obtaining the mixture, wherein the mixture is malleable and cohesive.

This disclosure may also be said broadly to consist in the parts, elements and features referred to or indicated in the specification of the application, individually or collectively, and any or all combinations of any two or more said parts, elements or features, and where specific integers are mentioned herein which have known equivalents in the art to which this disclosure relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the disclosed embodiments. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 shows samples of the biocompatible modified starch particles dyed with toluidine blue and overlaid with PBS, immediately after set-up.

FIG. 2 shows samples of the biocompatible modified starch particles dyed with toluidine blue and overlaid with PBS, after incubation overnight.

FIG. 3 shows a micrograph of the liquid from FIG. 2 at 50× magnification.

FIG. 4 shows micrographs of the biocompatible modified starch particles at 50× magnification, exposed to varying levels of FBS or BSA after 24 hours of incubation.

FIG. 5 shows the concentration of protein released from mixtures of bone dust (BD), a hydrogen delivery matrix (HDM) comprising the biocompatible modified starch particles, and bovine lactoferrin (bLF). The bars show samples after 24 hours (left, white bar), 48 hours (middle, red bar), and 72 hours (blue, right bar). All data shown are the mean of three replicates, with error bars showing the 95% confidence interval.

### DETAILED DESCRIPTION OF THE INVENTION

The present disclosure is based on the finding that biocompatible modified starch particles can be used as a hydrogel delivery matrix to promote healing of wounded tissue.

Accordingly, in a first aspect, the disclosure provides a method of delivering a biological material to a wounded tissue of a patient, the method comprising:
a. combining:
   i. a composition comprising biocompatible modified starch particles, wherein the starch particles are modified by carboxylation or hydroxylation of glucose units in the starch, and
   ii. the biological material
to form a mixture, and
   applying the mixture to the wounded tissue of the patient.

In a second aspect, the disclosure provides a method of delivering a biological material to a wounded tissue of a patient, the method comprising:
applying a mixture to the wounded tissue of the patient, the mixture having been obtained by combining to form a mixture,
   a. a composition comprising biocompatible modified starch particles, wherein the starch particles are modified by carboxylation or hydroxylation of glucose units in the starch, and
   b. the biological material.

### Definitions

The term “biological fluid” as used in this specification refers to fluids that are obtained from a biological source. Exemplary biological fluids include, but are not limited to, blood, blood plasma, lymph, spinal fluid, mucosal tissue secretions, interstitial fluid, synovial fluid, and saliva.

The term “bone dust” as used in this specification refers to a mixture of bone particles, blood products, fibrous tissue, and fluids. Bone dust is created when burrs are used during surgical procedures to remove unwanted bone tissue, such as during spinal fusion surgery. Alternatively, bone dust can be harvested from other sites, such as the iliac crest. The term is not intended to be limited to substances with a particular consistency. Bone dust typically resembles pâté in consistency, although this can vary depending on how much blood, plasma, biological fluids, and other fluids is present. Bone dust can be collected during surgery, for example by using an inline suction trap. It is recommended that the burr tip is constantly cooled during use to minimise the risk of thermal damage to the bone dust, for example by saline lavage.

The terms “cohesive”, “cohesiveness” and the like, as used in this specification, refer to the ability of a composition to maintain its shape while immersed in normal saline or water for a minimum of one minute.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The terms “malleable”, “malleability” and the like, as used in this specification, refer to the ability of a composition to be moulded into different shapes without visible cracks.

The term “patient” as used in this specification refers to any animal having a wound in need of healing. Animal patients include mammals, particularly humans, livestock animals such as cows, sheep, pigs and goats, and companion animals such as horses, dogs and cats. In a preferred embodiment, the patient is a human.

The term “promoting tissue healing” as used in this specification means an increase in tissue healing compared to untreated tissue, such as faster healing and/or improved quality of healing. This can include, for example, faster tissue union, improved consolidation, increased formation of new non-scar tissue, reduced formation of scar tissue, increased strength and/or flexibility of the healed wound, reduced inflammation, reduced risk of wound reopening, and/or reduced risk of infection.

The term “saline” as used in this specification refers to an aqueous solution comprising water and one or more salts. Preferably the one or more salts comprise sodium chloride. Saline solutions are well known in the art and are used in medicine for a wide variety of purposes including wound irrigation and intravenous administration. It will be appreciated that the salts used, and the concentration thereof, can vary according to the intended use. One commonly used saline, referred to as “normal saline”, comprises 0.90% sodium chloride by weight in pure water. Preferably the saline is sterile isotonic saline. “Isotonic” in this context means having an osmolarity that is similar to the osmolarity of human blood.

The term “wounded tissue” as used in this specification includes tissue that has been wounded in any way and by any mechanism. It includes, for example, wounds due to accident or injury, wounds due to disease or infection, or due to treatment of a disease or infection, and sites of surgical intervention. In some embodiments, the wounded tissue can be a combination of one or more of these, for example a site of surgical intervention to repair or treat a wound due to accident, injury, disease, or infection.

### Methods

In one aspect, the disclosure provides a method of delivering a biological material to a wounded tissue of a patient, the method comprising:
a. combining:
   i. a composition comprising biocompatible modified starch particles, wherein the starch particles are modified by carboxylation or hydroxylation of glucose units in the starch, and
   ii. the biological material
   to form a mixture, and
b. applying the mixture to the wounded tissue of the patient.

In another aspect, the disclosure provides a method of delivering a biological material to a wounded tissue of a patient, the method comprising:
applying a mixture to the wounded tissue of the patient, the mixture having been obtained by combining to form a mixture,
   a. a composition comprising biocompatible modified starch particles, wherein the starch particles are modified by carboxylation or hydroxylation of glucose units in the starch, and
   b. the biological material.

The combining can be performed by any suitable means known in the art, such as by stirring, shaking, or vortexing. In some embodiments, the combining takes place immediately prior to step (b).

In some embodiments, the biological material comprises a biological fluid. In some embodiments, the biological material comprises one or more tissue fragments. In some embodiments, the biological material comprises both a biological fluid and one or more tissue fragments.

In a preferred embodiment, the tissue fragments comprise autogenous tissue fragments. In some embodiments, the method further comprises the step of harvesting autogenous tissue fragments from the patient.

In another embodiment, the tissue fragments comprise allogenic tissue fragments. In some embodiments, the method further comprises the step of harvesting allogenic tissue fragments from a human donor.

In some embodiments the combining comprises combining:
a. a composition comprising biocompatible modified starch particles, wherein the starch particles are modified by carboxylation or hydroxylation of glucose units in the starch,
b. the biological material, and
c. a liquid.

In some embodiments, step (a) comprises combining the composition defined in (i), the one or more tissue fragments defined in (ii), and (iii) biological fluid and/or sterile fluid. In a preferred embodiment, the sterile fluid is sterile isotonic saline.

In various embodiments, the mixture forms a gel, putty, or paste. Preferably, the gel, putty, or paste is malleable and cohesive. The skilled person can manipulate the consistency of the mixture (i.e. the malleability and cohesiveness) by altering the ratio of biocompatible modified starch particles to fluid, for example by combining different amounts of biological fluid and/or sterile fluid. It will be appreciated that the one or more tissue fragments may be present in varying amounts of fluid, including varying amounts of biological fluid and/or varying amounts of other fluid (such as sterile saline used to lavage the burr tip when harvesting bone dust). Therefore, to achieve the desired malleability and cohesiveness, varying amounts of fluid may be added.

Without wishing to be bound by theory, it is believed that the mixture formed provides easier handling by a surgeon and allows the tissue fragments to be better placed at the wound site. The mixture holds the tissue fragments in place and conforms to the wound surface(s). The mixture allows bioactive molecules to diffuse out of the mixture to act at the wound site. Such bioactive molecules can include anabolic factors, growth factors, anti-inflammatory factors, and the like. The bioactive molecules can originate from the biological material, such as the tissue fragments, and/or the biological fluid. Alternatively, or additionally, one or more bioactive molecules and/or one or more therapeutic agents can be combined with or incorporated into the composition defined in (i).

The mixture also allows cells to migrate out of the biological material, and/or from the wound site into the gel. The gel may form a scaffold for the formation of new tissue. The mixture is bio-absorbable and will gradually degrade over time due to the action of digestive enzymes such as amylase.

### Compositions

In one aspect, the disclosure provides a composition comprising:
a. biocompatible modified starch particles;
b. optionally a liquid; and
c. a biological material;
wherein the composition is malleable and cohesive.

In another aspect, the disclosure provides a composition comprising:
a. biocompatible modified starch particles;
b. optionally a liquid; and
c. a biological material;
wherein the weight ratio of biocompatible modified starch particles to saline and/or biological material is from 1:1 to 1:50, preferably from 1:5 to 1:15.

In one embodiment, the liquid comprises saline.

In various embodiments, the biological material can be of any type described in the section “Biological material” below. In some embodiments, the biological material comprises a biological fluid and/or one or more tissue fragments. In a preferred embodiment, the tissue fragments are autogenous graft material.

In some embodiments, the composition is malleable and/or cohesive. In a preferred embodiment, the composition is both malleable and cohesive.

The malleability and cohesiveness of the compositions can be adjusted by varying the ratio of biocompatible modified starch particles to saline and/or biological fluid. For example, a composition with a relatively higher proportion of biocompatible modified starch particles will form a firmer composition (i.e. will be less malleable and more cohesive), while a lower proportion of biocompatible modified starch particles will form a softer composition (i.e. will be more malleable and less cohesive). A composition with too high a proportion of biocompatible modified starch particles will be crumbly, may contain unhydrated starch particles, and will tend to crack or break when moulded, while a composition with too low a proportion of biocompatible modified starch particles will remain fluid or semi-fluid and be unable to hold a shape.

The skilled person will appreciate that the ratio of biocompatible modified starch particles to saline and/or biological fluid can be adjusted to suit the intended application. Furthermore, the ratio can be adjusted to compensate for variability in the biological material. For example, if the biological material comprises a fluid, such as a biological fluid, a higher amount of biocompatible modified starch particles may be required to achieve a malleable and cohesive consistency. Preferably the composition has the consistency of a putty or paste, such as toothpaste or modelling clay.

In some embodiments, the composition has a viscosity at 25° C. of at least 50 mPa·s, such as at least 100, at least 500, at least 1,000, at least 2,000, at least 3,000, at least 4,000, at least 5,000, at least 7,500, at least 10,000, at least 20,000, at least 30,000, at least 40,000, at least 50,000, at least 60,000, at least 70,000, at least 80,000, at least 90,000, at least 100,000, at least 10⁶, at least 10⁷, at least 10⁸, at least 10⁹, at least 10¹⁰, or at least 10¹¹ mPa·s. Preferably, the composition has a viscosity at 25° C. of at least 30,000 mPa·s.

In some embodiments, the composition has a viscosity at 25° C. of from 50 to 10¹¹ mPa·s, such as from 1,000 to 10¹¹, from 5,000 to 10¹¹, from 10,000 to 10¹¹, from 20,000 to 10¹¹, from 30,000 to 10¹¹, from 40,000 to 10¹¹, from 50,000 to 10¹¹, from 60,000 to 10¹¹, from 70,000 to 10¹¹, from 80,000 to 10¹¹, from 90,000 to 10¹¹, from 100,000 to 10¹¹, from 10⁶ to 10¹¹, from 10⁷ to 10¹¹, from 10⁸ to 10¹¹, from 10⁹ to 10¹¹, from 10¹⁰ to 10¹¹, 50 to 10¹⁰, such as from 1,000 to 10¹⁰, from 5,000 to 10¹⁰, from 10,000 to 10¹⁰, from 20,000 to 10¹⁰, from 30,000 to 10¹⁰, from 40,000 to 10¹⁰, from 50,000 to 10¹⁰, from 60,000 to 10¹⁰, from 70,000 to 10¹⁰, from 80,000 to 10¹⁰, from 90,000 to 10¹⁰, from 100,000 to 10¹⁰, from 10⁶ to 10¹⁰, from 10⁷ to 10¹⁰, from 10⁸ to 10¹⁰, from 10⁹ to 10¹⁰, 50 to 10⁹, such as from 1,000 to 10⁹, from 5,000 to 10⁹, from 10,000 to 10⁹, from 20,000 to 10⁹, from 30,000 to 10⁹, from 40,000 to 10⁹, from 50,000 to 10⁹, from 60,000 to 10⁹, from 70,000 to 10⁹, from 80,000 to 10⁹, from 90,000 to 10⁹, from 100,000 to 10⁹, from 10⁶ to 10⁹, from 10⁷ to 10⁹, from 10⁸ to 10⁹, 50 to 10⁸, such as from 1,000 to 10⁸, from 5,000 to 10⁸, from 10,000 to 10⁸, from 20,000 to 10⁸, from 30,000 to 10⁸, from 40,000 to 10⁸, from 50,000 to 10⁸, from 60,000 to 10⁸, from 70,000 to 10⁸, from 80,000 to 10⁸, from 90,000 to 10⁸, from 100,000 to 10⁸, from 10⁶ to 10⁸, from 10⁷ to 10⁸, 50 to 10⁷, such as from 1,000 to 10⁷, from 5,000 to 10⁷, from 10,000 to 10⁷, from 20,000 to 10⁷, from 30,000 to 10⁷, from 40,000 to 10⁷, from 50,000 to 10⁷, from 60,000 to 10⁷, from 70,000 to 10⁷, from 80,000 to 10⁷, from 90,000 to 10⁷, from 100,000 to 10⁷, from 10⁶ to 10⁷, 50 to 10⁶, such as from 1,000 to 10⁶, from 5,000 to 10⁶, from 10,000 to 10⁶, from 20,000 to 10⁶, from 30,000 to 10⁶, from 40,000 to 10⁶, from 50,000 to 10⁶, from 60,000 to 10⁶, from 70,000 to 10⁶, from 80,000 to 10⁶, from 90,000 to 10⁶, from 100,000 to 10⁶, 50 to 100,000, such as from 1,000 to 100,000, from 5,000 to 100,000, from 10,000 to 100,000, from 20,000 to 100,000, from 30,000 to 100,000, from 40,000 to 100,000, from 50,000 to 100,000, from 60,000 to 100,000, from 70,000 to 100,000, from 80,000 to 100,000, from 90,000 to 100,000, 50 to 90,000, such as from 1,000 to 90,000, from 5,000 to 90,000, from 10,000 to 90,000, from 20,000 to 90,000, from 30,000 to 90,000, from 40,000 to 90,000, from 50,000 to 90,000, from 60,000 to 90,000, from 70,000 to 90,000, from 80,000 to 90,000, 50 to 80,000, such as from 1,000 to 80,000, from 5,000 to 80,000, from 10,000 to 80,000, from 20,000 to 80,000, from 30,000 to 80,000, from 40,000 to 80,000, from 50,000 to 80,000, from 60,000 to 80,000, from 70,000 to 80,000, 50 to 70,000, such as from 1,000 to 70,000, from 5,000 to 70,000, from 10,000 to 70,000, from 20,000 to 70,000, from 30,000 to 70,000, from 40,000 to 70,000, from 50,000 to 70,000, from 60,000 to 70,000, 50 to 60,000, such as from 1,000 to 60,000, from 5,000 to 60,000, from 10,000 to 60,000, from 20,000 to 60,000, from 30,000 to 60,000, from 40,000 to 60,000, from 50,000 to 60,000, 50 to 50,000, such as from 1,000 to 50,000, from 5,000 to 50,000, from 10,000 to 50,000, from 20,000 to 50,000, from 30,000 to 50,000, from 40,000 to 50,000, 50 to 40,000, such as from 1,000 to 40,000, from 5,000 to 40,000, from 10,000 to 40,000, from 20,000 to 40,000, from 30,000 to 40,000, 50 to 30,000, such as from 1,000 to 30,000, from 5,000 to 30,000, from 10,000 to 30,000, or from 20,000 to 30,000 mPa·s. Preferably, the composition has a viscosity at 25° C. of from 30,000 to 10⁶ mPa·s

In one embodiment, the weight ratio of biocompatible modified starch particles to saline and/or biological fluid is in the range of about 1:1 to about 1:50. In various embodiments, the weight ratio is in the range of about 1:1 to about 1:40, about 1:1 to about 1:30, about 1:1 to about 1:25, about 1:1 to about 1:20, about 1:1 to about 1:18, about 1:1 to about 1:16, about 1:1 to about 1:14, about 1:1 to about 1:12, about 1:1 to about 1:10, about 1:1 to about 1:8, about 1:1 to about 1:6, about 1:1 to about 1:4, about 1:1 to about 1:2, about 1:2 to about 1:50, about 1:2 to about 1:40, about 1:2 to about 1:30, about 1:2 to about 1:25, about 1:2 to about 1:20, about 1:2 to about 1:18, about 1:2 to about 1:16, about 1:2 to about 1:14, about 1:2 to about 1:12, about 1:2 to about 1:10, about 1:2 to about 1:8, about 1:2 to about 1:6, about 1:2 to about 1:4, about 1:4 to about 1:50, about 1:4 to about 1:40, about 1:4 to about 1:30, about 1:4 to about 1:25, about 1:4 to about 1:20, about 1:4 to about 1:18, about 1:4 to about 1:16, about 1:4 to about 1:14, about 1:4 to about 1:12, about 1:4 to about 1:10, about 1:4 to about 1:8, about 1:4 to about 1:6, about 1:6 to about 1:50, about 1:6 to about 1:40, about 1:6 to about 1:30, about 1:6 to about 1:25, about 1:6 to about 1:20, about 1:6 to about 1:18, about 1:6 to about 1:16, about 1:6 to about 1:14, about 1:6 to about 1:12, about 1:6 to about 1:10, about 1:6 to about 1:8, about 1:8 to about 1:50, about 1:8 to about 1:40, about 1:8 to about 1:30, about 1:8 to about 1:25, about 1:8 to about 1:20, about 1:8 to about 1:18, about 1:8 to about 1:16, about 1:8 to about 1:14, about 1:8 to about 1:12, about 1:8 to about 1:10, about 1:10 to about 1:50, about 1:10 to about 1:40, about 1:10 to about 1:30, about 1:10 to about 1:25, about 1:10 to about 1:20, about 1:10 to about 1:18, about 1:10 to about 1:16, about 1:10 to about 1:14, about 1:10 to about 1:12, about 1:12 to about 1:50, about 1:12 to about 1:40, about 1:12 to about 1:30, about 1:12 to about 1:25, about 1:12 to about 1:20, about 1:12 to about 1:18, about 1:12 to about 1:16, about 1:12 to about 1:14, about 1:14 to about 1:50, about 1:14 to about 1:40, about 1:14 to about 1:30, about 1:14 to about 1:25, about 1:14 to about 1:20, about 1:14 to about 1:18, about 1:14 to about 1:16, about 1:16 to about 1:50, about 1:16 to about 1:40, about 1:16 to about 1:30, about 1:16 to about 1:25, about 1:16 to about 1:20, about 1:16 to about 1:18, about 1:18 to about 1:50, about 1:18 to about 1:40, about 1:18 to about 1:30, about 1:18 to about 1:25, about 1:18 to about 1:20, about 1:20 to about 1:50, about 1:20 to about 1:40, about 1:20 to about 1:30, or about 1:20 to about 1:25. Preferably, the weight ratio is from about 1:5 to about 1:15, more preferably about 1:10.

In a preferred embodiment, the weight ratio of biocompatible modified starch particles to saline and/or biological fluid is sufficient to fully hydrate the starch particles. Preferably, the hydrated starch particles do not contain any remaining dry powder.

In some embodiments, the composition has a biologically acceptable level of heavy metal contamination. A biologically acceptable level of heavy metal contamination can readily be determined by a person skilled in the art.

### Biocompatible Modified Starch

In one aspect, the present disclosure provides a method of delivering a biological material to a wounded tissue of a patient, the method comprising the application of a composition comprising biocompatible modified starch particles to a wounded tissue of a patient. The biocompatible modified starch particles are gradually degraded when applied to the wound surface of humans and/or animals, are able to promote tissue healing, release factors that promote tissue growth and repair, release cells that facilitate wound repair, hold the biological material such as tissue fragments in place at a wound site, and act as a scaffold for cell growth.

Starch is a polymeric carbohydrate consisting of glucose units joined by glycosidic bonds. At room temperature, raw starch is generally not soluble in water, nor does it readily absorb water. Raw starch normally absorbs water at temperatures above 60° C. and swells to an adhesive, translucent and colloidal solution. The biocompatible modified starch particles of the present disclosure are raw starch processed through physical and chemical modifications, resulting in physical and chemical changes which make its characteristics and properties suitable for the applications contemplated herein. Starch is generally classified by its origin, such as potato starch or corn starch, etc. There are two types of glucose chains in starch: amylose, a simple linear chain comprising α-D-glucose units bonded to each other through α(1→4) glycosidic bonds; and amylopectin, a branched chain of α-D-glucose units comprising α(1→4) and a(1→6) glycosidic bonds. The diameter range of starch grains varies depending on its source and is normally from 1 to 100 μm with an average diameter of 15 to 30 μm.

Natural raw starch is not useful in the methods and compositions of the disclosure because natural raw starch grains are small and light, and the gelation properties are unsatisfactory at room temperature. Accordingly, the starch particles useful in the methods and compositions of the disclosure are biocompatible modified starch particles.

Starch particles can be modified by a number of methods, leading them to acquire altered chemical and physical characteristics. Modification can include adding, removing, or rearranging chemical groups that change the structure of the raw starch molecular chain. Modified starch can be categorised primarily into physically modified starch, chemically modified starch, enzymatically modified starch, and naturally modified starch, according to the modification process used.

Physical modification is a process which produces modified starch with the desired properties and functions by physically changing the microcrystalline starch structure through heating, extrusion, and/or irradiation. In some embodiments, the physically modified starch is pre-gelatinised starch (a-starch), y-ray treated starch, microwave or high frequency radiation treated starch, mechanically milled starch, and/or steam treated starch.

Chemically modified starch is produced by processing the raw starch with chemical agents that change the molecular structure to achieve the desired modified starch properties. In some embodiments, the chemically modified starch is an acid modified starch, oxidized starch, maltodextrin, esterified starch, etherified starch, and/or grafted starch.

Enzymatically modified starch is produced by processing the raw starch with enzymes. In some embodiments, the enzymatically modified starch is α-cyclic dextrin, β-cyclic dextrin, γ-cyclic dextrin, maltodextrin, and/or amylopectin.

Naturally modified starch may possess the same properties as chemically modified starch by changing the structure of natural raw starch with a variety of breeding and genetic techniques.

The modified starch particles may require multi-modification of the raw starch to achieve the desired properties. In other words, two or more modifying methods may be used to produce the final, composite, modified starch.

The characteristics of the modified starch particles can further include the acquisition of hydrophilic groups in its molecular chains through the described modification processes.

In some embodiments, the biocompatible modified starch particles useful in the methods and compositions of the disclosure includes starch modified physically, chemically, naturally, or enzymatically, and starch modified repeatedly with at least one of the above methods or a combination of two or more of the above methods.

In one embodiment, the modified starch particles are modified by irradiation, mechanical, and steam modification.

Physically modified starch, for example, a pre-gelatinised starch treated solely with spray drying or irradiation process, is remarkably safe as a bio-absorbable delivery matrix material since it is not treated with any chemical agents.

Pre-gelatinised starch can be modified by an extrusion process, roller drying, and a spray drying process. After heating a raw starch with a measured amount of water, the starch granules swell to a pasty substance, the regularly arranged micelles of starch are broken, crystallites disappear, and the resulting composition can be easily degraded by amylase enzymes. Pre-gelatinised starch is able to swell and/or dissolve in cold or room temperature water and form an adhesive paste whose retrogradation is lower than that of raw starch, affording easier handling during the production process. Raw starch can be pre-gelatinised through solely a physical modification process without adding any chemical agents and becomes a carrier matrix material with enhanced hydrophilic and adhesive properties.

The pre-gelatinised starch is safe, non-toxic, and has no adverse side effects. The pre-gelatinised starch is readily degraded and metabolized by enzymes in the body. The described pre-gelatinised material is safe and biocompatible.

The process of chemically modifying starch can include acidolysis, oxidation, esterification, etherification, cross-linking, and/or chemical agent grafting. The chemically modifying process can also comprises two or more of the above processes, or one of the above processes performed at least twice.

By adding functional groups to the raw starch glucose units with chemical agents, e.g. by carboxylation or hydroxylation, the starch gains hydrophilic groups in its molecular structure and obtains improved hydrophilic properties. By using bifunctional or polyfunctional chemical agents to cross-link the raw starch macromolecules, or by grafting external macromolecular hydrophilic groups to the raw starch, the starch acquires enhanced hydrophilic properties and viscosity/adhesiveness in a water solution. The viscosity of modified starch relates to the raw starch origin and the degree of substitution of external and the cross-linked or grafted functional groups, etc. When combined with tissue fragments, biological fluid, and/or saline solution, the hydrophilic and adhesive properties of the prescribed modified starch will produce a gel that can be easily handled and introduced into a wound. In addition, the interaction between the tissue fragments and the gelled modified starch matrix will cause the gelled composition to hold the tissue fragments in place within the wound defect, resulting in improved wound healing.

In various embodiments, the biocompatible modified starch particles useful in methods and compositions of the present disclosure contain one or more of pre-gelatinised starch, acid modified starch, dextrin, oxidized starch, esterified starch, etherified starch, or cross-linked starch. In a preferred embodiment, the biocompatible modified starch particles are etherified and/or cross-linked starch. In a more preferred embodiment, the biocompatible modified starch particles comprise cross-linked carboxymethyl starch or a pharmaceutically acceptable salt thereof.

The main physical parameters of the modified starch used in the methods and compositions of the present disclosure are provided below:

In some embodiments, the biocompatible modified starch useful in the methods and compositions of the present disclosure has a weight-averaged molecular weight over 15 kDa, preferably over 100 kDa, more preferably over 200 kDa, most preferably over 300 kDa. In some embodiments, the biocompatible modified starch has a weight-averaged molecular weight from 100 kDa to 1000 kDa, preferably from 150 to 750 kDa, more preferably from 200 to 500 kDa, most preferably from 290 to 400 kDa.

In some embodiments, the biocompatible modified starch useful in the methods and compositions of the present disclosure has a water absorbency capacity not lower than one time its weight (i.e. 1 gram of the biocompatible modified starch can absorb 1 gram or more of water), preferably 1-500 times its own weight, more preferably 5-100 times its own weight, more preferably 10-50 times its own weight, more preferably 20-30 times its own weight, most preferably about 23 times its own weight.

In one embodiment, the biocompatible modified starch particles useful in the methods and compositions of the present disclosure comprise particles with a diameter in the of range of 1-1000 μm, preferably 1-500 μm, more preferably 5-200 μm, most preferably 10-100 μm. In a preferred embodiment, at least 95% of the starch particles have diameters in the of range of 1-1000 μm, preferably 1-500 μm, more preferably 5-200 μm, most preferably 10-100 μm.

In a preferred embodiment, the biocompatible modified starch particles useful in the methods and compositions of the present disclosure comprise a sodium salt of cross-linked partly O-carboxymethylated starch.

Sodium carboxymethyl starch is a polymer of linear structure as expressed in the following formula:

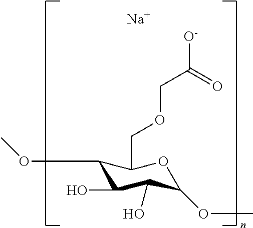

The starch does not need to be completely modified, i.e. it may contain a mixture of modified and unmodified subunits.

Modified starches such as carboxymethyl starch (CMS) and hydroxyethyl starch are used clinically as plasma substitutes. These modified starches exhibit excellent biocompatibility and safety with no toxic side effects when employed in the human circulatory system.

In a preferred embodiment, the biocompatible modified starch is cross-linked. Cross-linking can be performed by any applicable method known in the art, such as cross-linking using chemical cross-linking agents, enzymatic cross-linking, and/or cross-linking by physical treatment.

In some embodiments, the biocompatible modified starch particles useful in methods and compositions of the present disclosure are made by an agglomeration process and pellet fabrication. Normally, modified starch particle dimensions are relatively small and light and may need to agglomerate into larger sizes and heavier weights which can readily disperse into biological fluid and/or saline to generate a malleable gel to achieve an optimal healing outcome. The agglomeration process may not be necessary for large sized modified starch particles such as grafted starch or cross-linked starch.

Because pre-agglomerated modified starch particles are small and lightweight, they readily form a colloid on the surface of liquids. This can affect dispersion and gelation by preventing water molecules from further dispersing to other starch particles. Accordingly, in some embodiments, agglomeration processing technologies in the food and pharmaceutical industries are used to accumulate microscopic modified starch particles in the general 5-50 μm diameter range, creating clinically applicable particles with a diameter range of 30-500 μm. Modified starch particles produced by the process disclosed above exhibit rapid water absorption, strong hydrophilic properties and rapid dispersion in fluid to achieve a composition with improved properties.

In some embodiments, the biocompatible modified starch particles are non-porous. In some embodiments, the biocompatible modified starch particles are substantially devoid of a microporous surface. Certain known particles (such as the microporous polysaccharide hemospheres described in U.S. Pat. No. 6,060,461) rely on the microporous properties described therein to achieve their hemostatic function. In contrast, it is anticipated that the biocompatible modified starch particles described herein are useful in the methods described herein, without necessarily being porous and/or having a microporous surface.

The biocompatible modified starch particles described herein do not necessarily possess the microporous structure described in U.S. Pat. No. 6,060,461. The modification processes used to produce the biocompatible modified starch particles may be selected to produce particles with the desired properties, without requiring porosity and/or a microporous surface. These modification processes can include the degree of substitution, the ratios of amylopectin to amylose, the functional groups added, and/or the amount of cross-linking.

### Biological Material

In some embodiments, the modified starch particles are combined with biological material before being applied to the wounded tissue. The biological material may comprise a biological fluid and/or one or more tissue fragments.

The biological fluid may be any suitable biological fluid such as blood, blood plasma, lymph, spinal fluid, mucosal tissue secretions, interstitial fluid, synovial fluid, and/or saliva.

The tissue fragments can be any sort of tissue fragments suitable for implantation into the patient. Types of tissue fragments can include bone tissue fragments, cartilage tissue fragments, and/or connective tissue fragments. Preferably, the tissue fragments are of the same type as the wounded tissue, for example, bone fragments for a bone wound, cartilage fragments for a cartilage wound, and so on. In one embodiment, the tissue fragments comprise bone dust. In another embodiment, the tissue fragments comprise cartilage tissue.

It will be appreciated that the biological material may comprise a mixture of both biological fluid and one or more tissue fragments. For example, tissue fragments may be present in a biological fluid such as blood or lymph.

Sources of biological material, such as biological fluid and/or tissue fragments can include autogenous graft material, allogenic graft material, xenogeneic graft material, and/or recombinant graft material.

Autogenous graft material is material harvested from the same patient that will receive the graft. Autogenous bone grafts can include cancellous bone, cortical bone, and/or a mixture of these materials. Autogenous graft material can be harvested from a site that is separate from the implant site, such as the iliac crest, femur, or any other suitable site. Alternatively, the graft material can be harvested from the surgical site, and can include material that is removed during surgery, such as bone dust. Autogenous graft material is the preferred type of tissue fragments, as it is inherently histocompatible and avoids the potential for transmission of viral and bacterial diseases, however other sources of graft material may be useful in certain circumstances, such as when an autogenous source cannot provide sufficient material.

Allogenic graft material (also known as allograft material) is material harvested from a donor, such as dead bone harvested from a human cadaver. Allograft material can be treated to remove immunogenic components, using techniques known in the art. For example, allograft material can be demineralised by acid treatment to produce demineralised bone matrix (DBM). This process removes calcium and phosphate from the material, leaving behind the extracellular matrix which predominantly consists of type I collagen and non-structural proteins. DBM is typically less immunogenic than untreated allograft bone material.

Xenogeneic graft material (also known as xenograft material) is material harvested from a different species than the intended recipient. For example, xenograft material for use in a human patient could be harvested from a cow or pig. Xenogeneic graft material usually requires processing to reduce the potential for immune rejection and/or the transmission of bacterial or viral diseases, as is known in the art.

It is also envisaged that different sources of biological material could be combined. For example, in one embodiment, autogenous graft material could be supplemented with allogenic graft material and/or xenogenic graft material. As described above, this may be desirable, for example, in circumstances where there is insufficient allograft material available.

Recombinant graft material is material produced recombinantly, for example by transgenic bacteria, yeasts, fungi, and/or mammalian cell culture. Recombinant graft material can include human proteins such as bone morphogenic proteins (BMPs). Recombinant graft material may be used alone or may be used in combination with one or more other source of tissue fragments as described above. In some embodiments, the method of delivering a biological material to a wounded tissue in a patient comprises combining modified starch particles with at least one recombinant graft material to form a first mixture; harvesting autogenous tissue fragments from the patient and/or harvesting allogenic tissue fragments from a human donor; combining the autogenous tissue fragments and/or the allogenic tissue fragments with the first mixture to form a second mixture; and applying the second mixture to the patient.

The tissue fragments can be obtained using any of a variety of conventional techniques, such as for example, by biopsy or other surgical removal. Preferably, the tissue sample is obtained under aseptic conditions. The tissue fragments may be present in a biological fluid. The particle size of each tissue fragment can vary, for example, the tissue size can be in the range of about 0.1-3 mm³, in the range of about 0.5-1 mm³, in the range of about 1-2 mm³, or in the range of about 2-3 mm³, but preferably the tissue particle is less than 1 mm³.

In one embodiment, the biological material, such as the tissue fragments, are used without further processing. In an alternative embodiment, the biological material, such as the tissue fragments, are subjected to further processing before combining with the modified starch particles. This processing can include washing, mincing, grinding, milling, freeze-drying, demineralising, and/or treatment with chemical agents, such as acids, bases, and/or surfactants. In an optional embodiment, the minced tissue fragments may be contacted with a matrix-digesting enzyme to facilitate cell migration out of the extracellular matrix surrounding the cells. The enzymes are used to increase the rate of cell migration out of the extracellular matrix and into the gelled modified starch material. Suitable matrix-digesting enzymes that can be used include, but are not limited to, collagenase, chondroitinase, trypsin, elastase, hyaluronidase, peptidase, thermolysin, and other proteases.

In one embodiment, autogenous biological material (such as biological fluid and/or tissue fragments) is harvested from a patient, combined with the modified starch particles to form a mixture, and applied to the wounded tissue of the patient. In another embodiment, allogenic biological material (such as biological fluid and/or tissue fragments) are harvested from a donor, combined with the modified starch particles to form a mixture, and applied to the wounded tissue of the patient. In various embodiments, the starch particles form a gel when combined with the biological material and/or sterile saline solution.

In a preferred embodiment, the biological material has cells incorporated therein or added thereto. Suitable cell types include, but are not limited to, osteocytes, osteoblasts, osteoclasts, fibroblasts, stem cells, pluripotent cells, chondrocyte progenitors, chondrocytes, endothelial cells, macrophages, leukocytes, adipocytes, monocytes, plasma cells, mast cells, umbilical cord cells, stromal cells, mesenchymal stem cells, epithelial cells, myoblasts, tenocytes, ligament fibroblasts, neurons, and bone marrow cells. In a preferred embodiment, the cells can migrate out of the biological material and into the gelled starch matrix. In one embodiment, the biological material contains at least one cell capable of migrating out of the tissue fragments. Preferably, the biological material contains an effective amount of cells that can migrate out of the biological material.

In a preferred embodiment, the biological material has growth factors and/or anabolic cytokines incorporated therein or added thereto. In one embodiment, the growth factors comprise bone morphogenetic proteins (BMPs), fibroblast growth factor (FGF), insulin-like growth factors (IGFs), platelet-derived growth factor (PDGF), transforming growth factor-β (TGF-β), and/or vascular endothelial growth factor (VEGF). In one embodiment, the anabolic cytokines comprise interleukin 1-β (IL-1β) and/or interleukin 6 (IL-6). In a preferred embodiment, the growth factors and/or anabolic cytokines can diffuse out of the biological material and into the wound, promoting tissue healing.

In one embodiment, the modified starch particles are combined with biological fluid and/or saline in a weight ratio sufficient to obtain a gel with workable consistency. In one embodiment, the weight ratio of starch particles to other components is in the range of about 1:1 to about 1:100. In various embodiments, the weight ratio is in the range of about 1:1 to about 1:90, such as about 1:1 to about 1:80, about 1:1 to about 1:70, about 1:1 to about 1:60, about 1:1 to about 1:50, about 1:1 to about 1:40, about 1:1 to about 1:30, about 1:1 to about 1:25, about 1:1 to about 1:20, about 1:1 to about 1:18, about 1:1 to about 1:16, about 1:1 to about 1:14, about 1:1 to about 1:12, about 1:1 to about 1:10, about 1:1 to about 1:8, about 1:1 to about 1:6, about 1:1 to about 1:4, about 1:1 to about 1:2, about 1:2 to about 1:80, about 1:2 to about 1:70, about 1:2 to about 1:60, about 1:2 to about 1:50, about 1:2 to about 1:40, about 1:2 to about 1:30, about 1:2 to about 1:25, about 1:2 to about 1:20, about 1:2 to about 1:18, about 1:2 to about 1:16, about 1:2 to about 1:14, about 1:2 to about 1:12, about 1:2 to about 1:10, about 1:2 to about 1:8, about 1:2 to about 1:6, about 1:2 to about 1:4, about 1:4 to about 1:80, about 1:4 to about 1:70, about 1:4 to about 1:60, about 1:4 to about 1:50, about 1:4 to about 1:40, about 1:4 to about 1:30, about 1:4 to about 1:25, about 1:4 to about 1:20, about 1:4 to about 1:18, about 1:4 to about 1:16, about 1:4 to about 1:14, about 1:4 to about 1:12, about 1:4 to about 1:10, about 1:4 to about 1:8, about 1:4 to about 1:6, about 1:6 to about 1:80, about 1:6 to about 1:70, about 1:6 to about 1:60, about 1:6 to about 1:50, about 1:6 to about 1:40, about 1:6 to about 1:30, about 1:6 to about 1:25, about 1:6 to about 1:20, about 1:6 to about 1:18, about 1:6 to about 1:16, about 1:6 to about 1:14, about 1:6 to about 1:12, about 1:6 to about 1:10, about 1:6 to about 1:8, about 1:8 to about 1:80, about 1:8 to about 1:70, about 1:8 to about 1:60, about 1:8 to about 1:50, about 1:8 to about 1:40, about 1:8 to about 1:30, about 1:8 to about 1:25, about 1:8 to about 1:20, about 1:8 to about 1:18, about 1:8 to about 1:16, about 1:8 to about 1:14, about 1:8 to about 1:12, about 1:8 to about 1:10, about 1:10 to about 1:80, about 1:10 to about 1:70, about 1:10 to about 1:60, about 1:10 to about 1:50, about 1:10 to about 1:40, about 1:10 to about 1:30, about 1:10 to about 1:25, about 1:10 to about 1:20, about 1:10 to about 1:18, about 1:10 to about 1:16, about 1:10 to about 1:14, about 1:10 to about 1:12, about 1:12 to about 1:80, about 1:12 to about 1:70, about 1:12 to about 1:60, about 1:12 to about 1:50, about 1:12 to about 1:40, about 1:12 to about 1:30, about 1:12 to about 1:25, about 1:12 to about 1:20, about 1:12 to about 1:18, about 1:12 to about 1:16, about 1:12 to about 1:14, about 1:14 to about 1:80, about 1:14 to about 1:70, about 1:14 to about 1:60, about 1:14 to about 1:50, about 1:14 to about 1:40, about 1:14 to about 1:30, about 1:14 to about 1:25, about 1:14 to about 1:20, about 1:14 to about 1:18, about 1:14 to about 1:16, about 1:16 to about 1:80, about 1:16 to about 1:70, about 1:16 to about 1:60, about 1:16 to about 1:50, about 1:16 to about 1:40, about 1:16 to about 1:30, about 1:16 to about 1:25, about 1:16 to about 1:20, about 1:16 to about 1:18, about 1:18 to about 1:80, about 1:18 to about 1:70, about 1:18 to about 1:60, about 1:18 to about 1:50, about 1:18 to about 1:40, about 1:18 to about 1:30, about 1:18 to about 1:25, about 1:18 to about 1:20, about 1:20 to about 1:80, about 1:20 to about 1:70, about 1:20 to about 1:60, about 1:20 to about 1:50, about 1:20 to about 1:40, about 1:20 to about 1:30, or about 1:20 to about 1:25. Preferably, the weight ratio is about 1:10.

### Surgery and Other Treatments

The site in need of delivery of a biological material may be any number of areas comprising tissue that is injured, damaged, eroded, brittle, or defective in some other way such that it would benefit from the delivery of a biological material at that site. In one embodiment, the site in need of delivery of a biological material is a bone. In an alternative embodiment, the site in need of delivery of a biological material is cartilage. The delivery of a biological material is envisaged to promote tissue growth, leading to the acceleration of tissue growth at that site in comparison with the rate of tissue growth seen in patients who are not subject to the delivery of the biological material. Preferably, the delivery of a biological material promotes healing.

In another aspect, the disclosure provides a method of treating a wounded tissue of a subject, the method comprising:
delivering a mixture to a site of a wounded tissue of the patient, the mixture having been obtained by combining to form a mixture,
   a. a composition comprising biocompatible modified starch particles, wherein the starch particles are modified by carboxylation or hydroxylation of glucose units in the starch, and
   b. a biological material.

In one embodiment, the method comprises treating a patient having a bone injury or condition requiring a bone graft, comprising administering to the patient a mixture comprising a composition comprising a) biocompatible modified starch particles, wherein the starch particles are modified by carboxylation or hydroxylation of glucose units in the starch, and b) a biological material.

In one embodiment, the method comprises delivering or administering the mixture to the patient in lumbar fusion surgery, maxillofacial surgery, oral surgery, dental surgery, or cranial surgery.

In accordance with another embodiment, the present disclosure provides methods of bone grafting. These methods include the steps of delivering the mixture to a subject having a medical condition requiring a bone graft at the site of the medical condition; and creating new bone growth in vivo. The methods of the present disclosure may be used to treat medical conditions including fractures; knee injuries; hip injuries; missing teeth replacement, tooth implants requiring bone for a dental implant; treatment of critical-sized bony defects; bone injuries or defects requiring a fusion procedure including, for example, foot, ankle, fingers, wrists, spinal fusion; or combinations thereof.

Thus, the site may be a site of injury. Alternatively, or additionally, the site may be a site of surgical intervention.

By “site of an injury” we include the meaning that the site may be the site of an injury such as the fracture of a bone or the tear of cartilage. By “site of surgical intervention” we include the meaning that the site may be a site of a surgical intervention such as the insertion of an implant into a bone. The site may also be a combination of both a site of injury and a site of surgical intervention. In other words, when the site is one of both an injury and a surgical intervention, this may be, for example, the placing of an implant at the site of a fracture. Alternative embodiments of such sites that fall within the intended scope of the present disclosure will be immediately apparent to a person of skill in the art.

The site may be a site requiring bone fusion or comprising damaged bone, eroded bone or bone defects. Such embodiments may also be found in combination with each other or with a site of injury or surgical intervention.

It is envisaged that a site where there is damaged and/or eroded bone may be more prone to injury, such as fragility fractures experienced by sufferers of conditions such as osteoporosis. Further, in patients where a site requires bone fusion, one may expect that that site may also be a site of injury, which injury may have led to the requirement for the fusion of a bone. For example, a spinal injury may call for the fusion of two vertebrae to stabilise the spine. Alternatively, it may be a site of other pathology, for example due to degeneration between vertebrae resulting in the site being treated by surgical fusion of the vertebrae.

By “site comprising bone defects” we include the meaning that the bone at that site has a defective composition or structure in comparison to healthy bone. Such defects may be congenital, or they may be acquired through injury or disease or other cases as would be well known to a person of skill in the art. That a site has “bone defects” or damaged bone may be assessed, for example, radiologically (e.g. by X-ray or by CT scan), as would be appreciated by a skilled person.

The disclosed embodiments may be useful in repairing damaged and/or eroded bone. By “damaged and/or eroded bone” we include the meaning that the bone has accumulated damage though environmental factors or genetic factors that have left the bone in a state of fragility and where the bone is weak and prone to fracture. Thus, the disclosed embodiments may be useful in repairing bone after osteomyelitis (infection of the bone) has damaged the bone. It is also envisaged that the disclosed embodiments will be useful in repairing bone damage after irradiation or chemotherapy, in patients with bone metastases of tumours or multiple myeloma. Congenital and other defects of bone may also be repaired using the methods and uses of the present disclosure.

It is envisaged that in any aspect of the present disclosure, the delivery of a biological material will aid in the promotion of tissue growth and/or healing the site. As indicated above, the site may be a site of injury and/or surgical intervention. It is expected that, when the site of injury or surgical intervention is bone, the site will comprise cut, eroded, damaged or broken bone. The promotion of bone formation is considered therefore to aid in healing fractures or fissures in the bone and in improving the strength, flexibility and/or quality of the bone and in fusing bone and/or repairing bone, as appropriate. The disclosed embodiments may be particularly useful in situations where rapid healing is a high priority.

Thus, in a preferred embodiment of the present disclosure, the site may be a site of injury and the injury may be a fracture of a bone.

The present disclosure is also considered to be useful in repairing less severely damaged bone, thus allowing the tissue layers or bone that were present at the site before the injury or surgical intervention occurred to be replenished. Such an embodiment is considered to be particularly useful after the insertion of an implant into the bone, where new bone formation is considered to enable the implant to adhere more securely than it would in the absence of the effects of the disclosed embodiments. Examples include fixation of screws and implants for joint replacement.

The disclosed embodiments may allow modulation of bone healing to accelerate as well as improve the quality of healing. This would allow for faster union and improved consolidation of the fracture or implant fixation. In the clinical scenario, there is a race between fracture union/consolidation and implant failure, especially in compromised bone as exemplified by fragility fractures. The disclosed embodiments are considered to promote union and consolidation, thereby reducing complications at the fracture or implant site and allow more rapid mobilisation of the patient.

In an embodiment of any aspect of the disclosure, the surgical intervention may be an osteotomy. By “osteotomy” we include any surgical procedure where bone is purposefully cut to shorten, lengthen or otherwise change its alignment. The present disclosure is envisaged to provide a means by which the healing process, after such a procedure, may be accelerated.

In an embodiment of any aspect of the present disclosure, it is considered that the promotion of bone formation at the site may aid in repairing bone at the site and/or accelerating bone formation at the site and/or increasing cortical bone volume and/or cortical bone mineral content and/or bone mineral density at the site and/or increasing mineralised volume of the healing bone and/or the mineralised bone volume fraction and/or tissue mineral density at the site and/or accelerating remodelling of the callus at the site, for example a fracture site, and/or accelerating remodelling of any newly formed bone at the site. Thus, the disclosed embodiments may increase the bone mineral density and/or bone volume and/or mature bone content at the site. It is further considered that the disclosed embodiments may lead to an improvement in the stiffness of the bone at a fracture site. Thus, the disclosed embodiments may aid in improving the strength of the newly formed bone and may reduce the likelihood of further fractures or other pathologies at that site.

In an alternative embodiment, the surgical intervention may be a procedure for inserting an implant into, around and/or adjacent to a bone. Alternatively, the surgical intervention may be for fixing an implant to a bone. Such implants may be selected from, but not limited to, the group comprising a joint replacement, a dental implant, a pin, a plate, a screw, an intradmedullary or intraosseous device. Exemplary joint replacements include hip replacements, knee replacements, shoulder replacements and elbow replacements. Dental implants may include implants into the mandible or maxilla to support crowns or other prosthetic dental structures or other implants as would be appreciated by a person of skill in the art. Further joints that may receive implants include the ankles, wrists, digits and spine. Pins and plates may be inserted to strengthen a bone or joint after an injury, such as a fracture. Promoting fixation may also be useful in osteointegrated implants, e.g. teeth, digits, facial prosthesis and hearing devices.

The methods of the present disclosure are therefore useful for promoting healing in a variety of surgeries, such as, for example, spinal fusion surgery, lumbar fusion surgery, maxillofacial surgery, oral surgery, dental surgery, cranial surgery, and/or cartilage repair surgery. Such surgeries can include: the repair of simple and compound fractures and non-unions; external and internal fixations; joint reconstructions such as arthrodesis; general arthroplasty; cup arthroplasty of the hip; femoral and humeral head replacement; femoral head surface replacement and total joint replacement; repairs of the vertebral column including spinal fusion and internal fixation; tumor surgery, e.g., deficit filing; discectomy; laminectomy; excision of spinal cord tumors; anterior cervical and thoracic operations; repairs of spinal injuries; scoliosis, lordosis and kyphosis treatments; intermaxillary fixation of fractures; mentoplasty; temporomandibular joint replacement; alveolar ridge augmentation and reconstruction; inlay osteoimplants; implant placement and revision; sinus lifts; cosmetic enhancement; etc. Specific bones which can be repaired using the methods of the invention include, but are not limited to: the ethmoid; frontal; nasal; occipital; parietal; temporal; mandible; maxilla; zygomatic; cervical vertebra; thoracic vertebra; lumbar vertebra; sacrum; rib; sternum; clavicle; scapula; humerus; radius; ulna; carpal bones; metacarpal bones; phalanges; ilium; ischium; pubis; femur; tibia; fibula; patella; calcaneus; tarsal and metatarsal bones.

An especially preferred use of the disclosed embodiments is to promote arthrodesis between vertebrae in spinal fusions in humans or other mammals, including for example interbody, posterior and/or posterolateral fusion techniques.

In addition, the methods of the disclosure can be used in conjunction with a load-bearing spinal or other orthopedic implant device (e.g. having a compressive strength of at least about 10000 N) such as a fusion cage, dowel, or other device having a pocket, chamber or other cavity for containing an osteoinductive and/or osteoconductive composition, and used in a spinal fusion such as an interbody fusion. In one illustrative example, the methods of the disclosure can be used in conjunction with a load-bearing interbody spinal spacer to achieve an interbody fusion.

In some embodiments, the site in need of promoting healing is cartilage.

Damage to cartilage, such as the cartilage that protects joints, can result from physical injury such as a result of trauma, sports or repetitive stresses (e.g., osteochondral fracture, secondary damage due to cruciate ligament injury), and/or from disease such as osteoarthritis, rheumatoid arthritis, aseptic necrosis, osteochondritis dissecans, or avascular necrosis. Thus, in some embodiments, the site in need of promoting healing is a cartilage injury resulting from trauma and/or disease.

In some embodiments, the compositions and methods of the disclosure are useful in non-surgical methods of treatment.

### Biologically Active Agents

In some embodiments, the composition used in the methods of the disclosure comprise biologically active agents. The biologically active agents can comprise one or more anabolic, osteogenic, osteoinductive, anti-inflammatory, antimicrobial, antibacterial, and/or antibiotic substances.

In certain embodiments, the osteoinductive substance can include one or more growth factors that is/are effective in inducing formation of bone. Desirably, the growth factor will be from a class of proteins known generally as bone morphogenic proteins (BMPs), and can in certain embodiments be recombinant human (rh) BMPs. These BMP proteins, which are known to have osteogenic, chondrogenic and other growth and differentiation activities, include rhBMP-2, rhBMP-3, rhBMP4 (also referred to as rhBMP-2B), rhBMP-5, rhBMP-6, rhBMP-7 (rhOP-1), rhBMP-8, rhBMP-9, rhBMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12, rhGDF-14. For example, BMP-2, BMP-3, BMP-4, BMP-5, BMP-6 and BMP-7, disclosed in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in U.S. Pat. No. 5,637,480; BMP-11, disclosed in U.S. Pat. No. 5,639,638, or BMP-12 or BMP-13, disclosed in U.S. Pat. No. 5,658,882, BMP-15, disclosed U.S. Pat. No. 5,635,372 and BMP-16, disclosed in U.S. Pat. Nos. 5,965,403 and 6,331,612. Other compositions which may also be useful include Vgr-2, and any of the growth and differentiation factors including those described in PCT applications WO94/15965; WO94/15949; WO95/01801; WO95/01802; WO94/21681; WO94/15966; WO95/10539; WO96/01845; WO96/02559 and others. Also useful in the present invention may be BIP, disclosed in WO94/01557; HP00269, disclosed in JP Publication number: 7-250688; and MP52, disclosed in PCT application WO93/16099. The growth factor may also be a LIM Mineralization Protein (LMP), including for example one or more of those disclosed in U.S. Pat. Nos. 6,858,431 and 7,045,614. The disclosures of all of these patents and applications are hereby incorporated herein by reference. Also useful in the present disclosure are heterodimers of the above and modified proteins or partial deletion products (biologically active fragments) thereof. These proteins can be used individually or in mixtures of two or more. rhBMP-2 or biologically active derivatives or fragments thereof that exhibit an ability to induce bone growth are preferred.

The BMP or other osteoinductive protein may be recombinantly produced. The BMP may be homodimeric, or may be heterodimeric with other BMPs (e.g., a heterodimer composed of one monomer each of BMP-2 and BMP-6) or with other members of the TGF-beta superfamily, such as activins, inhibins and TGF-beta 1 (e.g., a heterodimer composed of one monomer each of a BMP and a related member of the TGF-beta superfamily). Examples of such heterodimeric proteins are described for example in Published PCT Patent Application WO 93/09229, the specification of which is hereby incorporated herein by reference. The amount of osteogenic protein useful herein is that amount effective to stimulate increased osteogenic activity of infiltrating progenitor cells, and will depend upon several factors including the size, location, and nature of the defect being treated, and the particular protein being employed. In certain embodiments, the amount of osteogenic protein to be delivered to the implant site will be in a range of from about 0.05 to about 1.5 mg.

In some embodiments, the composition comprises biologically active agents in an effective amount to render the composition osteoinductive when implanted in a mammal, such as a human patient. In one embodiment, the methods of the disclosure comprises applying a composition comprising a bone morphogenic protein or other osteogenic protein at a level of about 0.6 milligrams per cubic centimetre (mg/cm³) of the composition to about 2 mg/cm³ of the composition, advantageously at a level of about 0.8 mg/cm³ to about 1.8 mg/cm³, to the patient.

In some embodiments, the osteoinductive substance can include a lactoferrin protein, polypeptide, or fragment thereof, and/or a mixture of any of these. The lactoferrin or polypeptide thereof can be isolated from mammalian milk, for example from cow's milk, or can be produced recombinantly, such as in bacteria, yeast, or fungi. Methods for the recombinant production of lactoferrin are known in the art. Some such methods are disclosed in PCT publication WO2003/082921, which is incorporated herein by reference. In one embodiment, the lactoferrin protein, polypeptide, or fragment is bovine lactoferrin. In an alternative embodiment, the lactoferrin protein, polypeptide, or fragment is human lactoferrin, such as recombinant human lactoferrin.

In some embodiments, the lactoferrin protein, polypeptide, or fragment is present in a concentration effective to stimulate the proliferation and/or differentiation of osteoblast-like cells and/or chondrocytes, reduce the rate of apoptosis of osteoblast-like cells, and/or inhibit osteoclastogenesis. In some embodiments, the lactoferrin protein, polypeptide, or fragment is present in a concentration of at least about 10 μg per g of the modified starch particles, such as at least about 15 μg, at least about 20 μg, at least about 30 μg, at least about 40 μg, at least about 50 μg, at least about 60 μg, at least about 70 μg, at least about 80 μg, at least about 90 μg, at least 100 μg, at least about 200 μg, at least about 300 μg, at least about 400 μg, at least 500 μg, at least 1000 μg, at least 2000 μg, at least 3000 μg, at least 4000 μg, or at least 5000 μg per g of the modified starch particles. In some embodiments, the lactoferrin protein, polypeptide, or fragment is present in a concentration of 10-5000 μg per g of the modified starch particles, such as 15-5000 μg, 20-5000 μg, 30-5000 μg, 40-5000 μg, 50-5000 μg, 60-5000 μg, 70-5000 μg, 80-5000 μg, 90-5000 μg, 100-5000 μg, 150-5000 μg, 200-5000 μg, 250-5000 μg, 300-5000 μg, 400-5000 μg, 500-5000 μg, 600-5000 μg, 700-5000 μg, 800-5000 μg, 900-5000 μg, 1000-5000 μg, 2000-5000 μg, 3000-5000 μg, 4000-5000 μg, 15-4000 μg, 20-4000 μg, 30-4000 μg, 40-4000 μg, 50-4000 μg, 60-4000 μg, 70-4000 μg, 80-4000 μg, 90-4000 μg, 100-4000 μg, 150-4000 μg, 200-4000 μg, 250-4000 μg, 300-4000 μg, 400-4000 μg, 500-4000 μg, 600-4000 μg, 700-4000 μg, 800-4000 μg, 900-4000 μg, 1000-4000 μg, 2000-4000 μg, 3000-4000 μg, 15-3000 μg, 20-3000 μg, 30-3000 μg, 40-3000 μg, 50-3000 μg, 60-3000 μg, 70-3000 μg, 80-3000 μg, 90-3000 μg, 100-3000 μg, 150-3000 μg, 200-3000 μg, 250-3000 μg, 300-3000 μg, 400-3000 μg, 500-3000 μg, 600-3000 μg, 700-3000 μg, 800-3000 μg, 900-3000 μg, 1000-3000 μg, 2000-3000 μg, 15-2000 μg, 20-2000 μg, 30-2000 μg, 40-2000 μg, 50-2000 μg, 60-2000 μg, 70-2000 μg, 80-2000 μg, 90-2000 μg, 100-2000 μg, 150-2000 μg, 200-2000 μg, 250-2000 μg, 300-2000 μg, 400-2000 μg, 500-2000 μg, 600-2000 μg, 700-2000 μg, 800-2000 μg, 900-2000 μg, 1000-2000 μg, 15-1000 μg, 20-1000 μg, 30-1000 μg, 40-1000 μg, 50-1000 μg, 60-1000 μg, 70-1000 μg, 80-1000 μg, 90-1000 μg, 100-1000 μg, 150-1000 μg, 200-1000 μg, 250-1000 μg, 300-1000 μg, 400-1000 μg, 500-1000 μg, 600-1000 μg, 700-1000 μg, 800-1000 μg, 900-1000 μg, 15-500 μg, 20-500 μg, 30-500 μg, 40-500 μg, 50-500 μg, 60-500 μg, 70-500 μg, 80-500 μg, 90-500 μg, 100-500 μg, 150-500 μg, 200-500 μg, 250-500 μg, 300-500 μg, 400-500 μg, 15-400 μg, 20-400 μg, 30-400 μg, 40-400 μg, 50-400 μg, 60-400 μg, 70-400 μg, 80-400 μg, 90-400 μg, 100-400 μg, 150-400 μg, 200-400 μg, 250-400 μg, 300-400 μg, 15-300 μg, 20-300 μg, 30-300 μg, 40-300 μg, 50-300 μg, 60-300 μg, 70-300 μg, 80-300 μg, 90-300 μg, 100-300 μg, 150-300 μg, 200-300 μg, 250-300 μg, 15-200 μg, 20-200 μg, 30-200 μg, 40-200 μg, 50-200 μg, 60-200 μg, 70-200 μg, 80-200 μg, 90-200 μg, 100-200 μg, 150-200 μg, 15-100 μg, 20-100 μg, 30-100 μg, 40-100 μg, 50-100 μg, 60-100 μg, 70-100 μg, 80-100 μg, or 90-100 μg per g of the modified starch particles.

Other therapeutic agents, such as therapeutic growth factors or substances may also be combined with the modified starch particles, especially those that can be used to stimulate bone formation. Examples of such agents include, but are not limited to, pharmaceutical agents, growth factors, cytokines, anabolic factors, osteoinductive factors, bone morphogenic proteins (BMPs), tissue growth enhancing agents, and/or viable cells. Specific examples include demineralized bone matrix, platelet-derived growth factors, insulin-like growth factors, cartilage-derived morphogenic proteins, statins, and transforming growth factors, including TGF-αand TGF-β.

The osteoinductive proteins and/or other biologically active agents to be used in the disclosed embodiments can be provided in liquid formulations, for example buffered aqueous formulations. In certain embodiments, such formulations are mixed with, received upon and/or within, or otherwise combined with a dried modified starch material, which is then manipulated to prepare a malleable osteoinductive material to be used in a method of the invention.

As further enhancements of the compositions of the disclosed embodiments, those skilled in the art will readily appreciate that other biologically active agents can be incorporated into the compositions. Such additional agents include, but are not limited to, microglobulin-beta, antibiotics, antifungal agents, steroids and non-steroidal anti-inflammatory compounds.

### EXAMPLES

### 1. Example 1—Release of Small Molecule Substances from Biocompatible Modified Starch Gel

### 1.1 Materials and Methods

Biocompatible modified starch particles, in the form of crosslinked carboxymethyl starch particles, were used. Phosphate buffered saline (PBS) containing 137 mM NaCl, 2.7 mM KCl, 10 mM Na₂HPO₄, and 1.8 mM KH₂PO₄ was used. All chemicals were obtained from Sigma-Aldrich.

Foetal bovine serum (FBS) was obtained from Gibco. 24-well cell culture plates (Gibco) were used.

### 1.2 Results

To determine effective starch to fluid ratios, different volumes of phosphate buffered saline (PBS) were added to a small sample of the biocompatible modified starch particles. 26 mg of the starch particles was weighed out in an Eppendorf tube and transferred to a small petri dish, and varying volumes of PBS was added.

At a 1:1 ratio of starch to fluid (26 mg of starch particles+26 μl PBS), too much dry starch powder remained. At a 1:5 ratio (26 mg of starch particles+130 μl PBS) a stiff gel formed, however this was too stiff to easily work with. At a 1:10 ratio (26 mg of starch particles+260 μl PBS) a gel with a good working consistency formed. This ratio was used for further experiments.

Samples of the biocompatible modified starch particles (27.0-28.0 mg) were added to six individual wells of a 24-well plate and 10× their weight of a toluidine blue solution was added. Each well was gently mixed with a 100 μl pipette tip and spread evenly over the entire bottom of the well, forming a ˜3 mm thick gel. A 1-ml layer of PBS was gently added to each well, and the results observed.

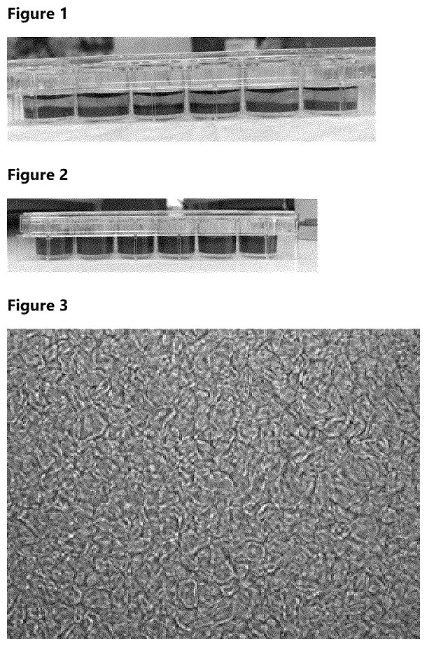

The wells initially showed two distinct phases, a deep blue-purple coloured gel at the bottom and a lighter-coloured PBS layer on top (FIG. 1). Over the course of an hour, the PBS layer became progressively darker blue as the toluidine blue was slowly released from the gel.

After incubation overnight, the gel appeared to be completely dissolved, and the liquid in the wells was a uniform blue colour (FIG. 2). Samples of the liquid was viewed under a microscope, and showed that it was full of small particles approximately 50-400 μm in diameter (FIG. 3).

### 1.3 Conclusion

This example shows that the biocompatible modified starch particles will form a gel with a good working consistency when dissolved in fluid in a ˜1:10 ratio of starch particles to fluid.

This example also shows that an indicator small molecule (toluidine blue) will slowly diffuse out of a starch gel over the course of several hours, and the starch gel will disperse when exposed to exposed to greater volumes of fluid. This supports the idea that osteogenic factors from bone dust, or other bioactive molecules incorporated into the starch (such as lactoferrin), will slowly diffuse out of the gel.

### 2. Example 2—Degradation of Biocompatible Modified Starch Particles by Amylase

### 2.1 Materials and Methods

Two wells of a 24-well plate were set up. For each well, 59 mg of the biocompatible modified starch powder was combined with 590 μl of PBS only (control) or PBS containing

Two wells of a 24-well plate were set up containing the biocompatible modified starch and PBS only (control) or PBS with 10% foetal bovine serum (FBS). The plates were incubated at 37° C. and inspected using a microscope at 24, 48, and 72 hours.

This experiment was repeated using varying concentrations of FBS (0%, 1%, 5%, 10%, 15%, and 20%) as well as 0.1% bovine serum albumin (BSA).

### 2.2 Results

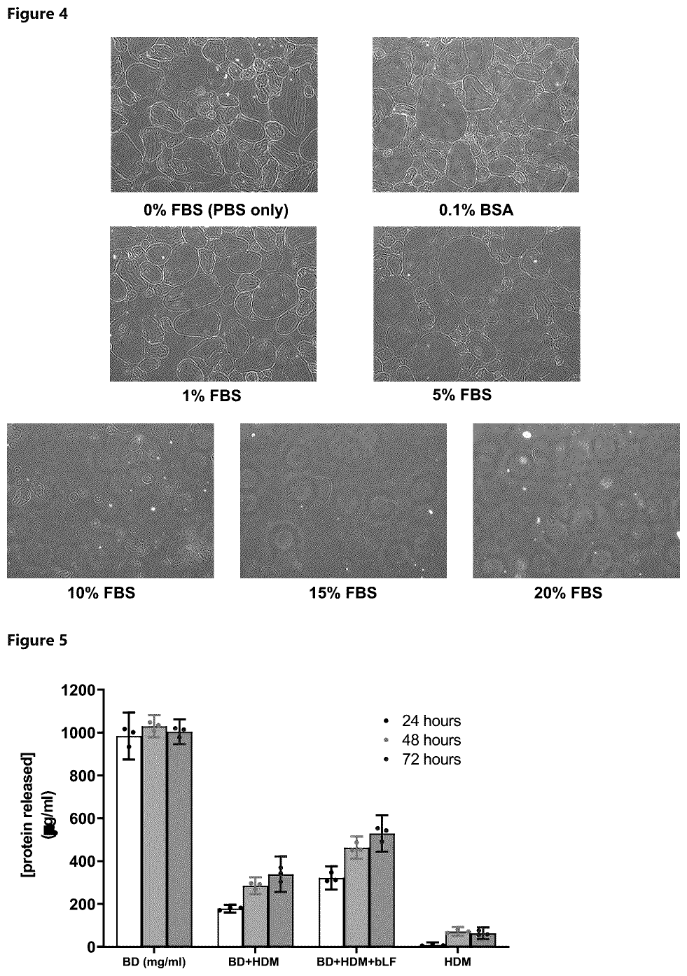

The effect of 10% FBS on the biocompatible modified starch particles was tested over the course of 72 hours. The visual inspection results of this are shown in Table 1, and micrographs taken at 24 hours are shown in FIG. 4.

TABLE 1
Visual inspection of biocompatible modified starch
particles in the presence or absence of 10% FBS.

| | 24 hours | 48 hours | 72 hours |
|---|---|---|---|
| Control (PBS | Lots of starch | No change in the | No change in |
| only) | particles in | number or size of | the number or |
| | the well | particles | size of particles |
| PBS + 10% FBS | Fewer particles, | A lot fewer | remaining |
| | and those | particles | Very few |
| | remaining changed | than at 24 | particles |
| | from a spheroid | hours, and | |
| | shape to | they appear | |
| | an ellipsoid shape, | fatter | |
| | like they were | and ‘thinner’ | |
| | being digested | | |

This experiment was repeated, varying the amount of FBS and BSA, as shown in Table 2. Micrographs of the particles at 24 hours are shown in FIG. 4.

TABLE 2
Visual inspection of biocompatible modified starch particles in
the presence of varying amounts of FBS or BSA.

| | 24 hours | 48 hours |
|---|---|---|
| Control (PBS only) | Lots of particles | Lots of particles |
| PBS + 0.1% BSA | Lots of particles, no visible | Lots of particles, |
| | difference to control | no visible |
| | | difference to control |
| PBS + 1% FBS | Slightly fewer particles than | Still a few particles |
| | control | present, but |
| | | much reduced |
| PBS + 5% FBS | Fewer particles than 1% FBS | Hardly any particles |
| PBS + 10% FBS | Even fewer particles | Hardly any particles |
| | than 5% FBS | |
| PBS +15% FBS | Even fewer particles | No particles present |
| | than 10% FBS | |
| PBS + 20% FBS | No particles present | No particles present |

### 2.3 Conclusion

This example shows that the biocompatible modified starch particles are slowly broken down over the course of several days by a component of FBS, presumable amylase enzymes. This effect is dependent on the amount of FBS present. This suggests that the biocompatible modified starch particles will be suitable for use as a hydrogel delivery matrix for tissue fragments (such as bone dust), as the gel will remain intact for several days, holding the tissue fragments at the wound site for long enough to begin the healing process, but will eventually be degraded by endogenous amylases.

### 3. Example 3—Compatibility of Starch Particles with ThinCert® Cell Culture Inserts

ThinCert® cell culture inserts (Greiner Bio-One Company) are often used to support in vitro cell cultures. We have previously shown that bone dust suspended above osteoblast cultures releases anabolic factors that increase osteoblast proliferation and up-regulates numerous osteoblastic genes integral to osteoblast differentiation, maturation and angiogenesis (Gao et al. 2018). The aim of this Example is to test the compatibility of the modified starch particles with this in vitro model.

### 3.1 Materials and Methods

In an initial experiment, 25 mg of the biocompatible modified starch mixed with 250 μl PBS was used, however this was found to cover the majority of the ThinCert® area, so smaller amounts would be required when combining the starch particles with bone dust.

ThinCert® cell culture inserts with a sieve size of 1 μm were set up over the wells of a 24-well cell culture plate. Solutions were added to the wells and inserts as set out in Table 3. The plates were incubated overnight at 37° C. and the results visualised the following day.

TABLE 3
Set-up of ThinCert ® cell culture inserts.

| Well | Well solution | ThinCert ® solution |
|---|---|---|
| Starch particles | 1 ml PBS | 11 mg starch particles + 110 |
| | | μl PBS + 100 μl toluidine |
| | | blue |
| PBS only, dye | 1 ml PBS | 121 μl PBS + 100 μl toluidine |
| in ThinCert ® | | blue |
| PBS only, | 1 ml PBS + 100 μl | 121 μl PBS |
| dye in well | toluidine blue | |
| Starch particles + | 1 ml PBS | 11 mg starch particles + 110 |
| FBS | | μl PBS + 100 μl toluidine |
| | | blue + 100 μl FBS |

### 3.2 Results

The effect of the biocompatible modified starch particles on dye diffusion through a ThinCert® membrane is shown in Table 4. Cell culture inserts without starch particles allowed the diffusion of dye in either direction, however the addition of the starch particles greatly reduced this, likely due to starch particles clogging the membrane pores. The addition of FBS allowed the dye to diffuse readily, likely due to amylase enzymes breaking down the starch particles.

TABLE 4
Effect of biocompatible modified starch particles on dye diffusion
through a ThinCert ® membrane.

| Well solution | ThinCert ® solution | Resμlt |
|---|---|---|
| PBS | starch particles + | Only a slight colour change |
| | PBS + toluidine blue | in the well solution |
| PBS | PBS + toluidine blue | Well solution very blue |
| PBS + | PBS | ThinCert ® solution very blue |
| toluidine blue | | |
| PBS | starch particles + | Well solution very blue |
| | PBS + toluidine | |
| | blue + FBS | |

### 3.3 Conclusion

This Example suggests that the modified starch particles can be used in tissue culture assays as previously described (Gao et al. 2018).

### 4. Example 4—ThinCert® Cell Culture Inserts with Bone Dust

### 4.1 Materials and Methods

Osteoblast culture assays will be set up as previously described (Gao et al. 2018). Briefly, 24-well tissue culture plates will be seeded with primary human osteoblasts in Dulbecco's Modified Eagle Medium (DMEM) supplemented with 5% foetal bovine serum (FBS) and 10 μg L-ascorbic acid-2-phosphate (AA2P), and incubated at 37° C. for 24 hours. Cells will then be growth-arrested by incubation in 0.1% BSA for 24 hours.

Tissue culture inserts containing samples or an empty control will be added to each well, and the plates incubated for a further 12 hours. The inserts will then be removed, and cell mitogenesis measured.

The samples to be tested will include:
1. an empty tissue culture insert (empty control),
2. 100 mg of bone dust (bone dust control),
3. 100 mg of bone dust mixed with 10 mg of the starch particles to form a gel,
4. 1, 10, or 100 μg of bovine lactoferrin in 100 μl of PBS,
5. 100 mg of bone dust mixed with 10 mg of the starch particles and 1, 10, or 100 μg of bovine lactoferrin.

### 4.2 Results

We expect that the empty tissue culture insert will show little or no proliferation of osteoblast cells, and the bone dust control will show increased proliferation as previously shown (Gao et al. 2018).

We expect that the combination of bone dust mixed with starch particles will show increased proliferation compared to the empty control. We expect that the gel will slowly degrade, allowing osteogenic factors from the bone dust to diffuse through the ThinCert® membrane. This process may be slowed, resulting in a controlled release of osteogenic factors over a longer time period than bone dust alone.

We expect that lactoferrin will show a dose-dependent increase in proliferation, and that the combination of bone dust, lactoferrin, and starch particles will show increased proliferation than any of these factors alone.

### 4.3 Conclusions

We expect that this Example will show that tissue fragments and/or biological fluid (such as bone dust) can be combined with the starch particles to form a gel. The gel will have improved properties such as gel-like consistency, allowing it to easily be placed within a wound and hold the bone dust in place. The starch will slowly biodegrade over time due to digestion by endogenous amylase enzymes, gradually releasing osteogenic factors from the bone dust and improving wound healing.

### 5. Example 5—Release of Proteins from HDM

### 5.1 Materials and Methods

Bone dust was prepared from trabecular/marrow bone as described in Gao eta((2018) and frozen until needed.

The frozen bone dust (BD) was thawed and 100 mg aliquots were added to twelve 5-ml vials. Biocompatible modified starch particles (hydrogel delivery matrix, HDM), bovine lactoferrin (bLF), and phosphate buffered saline (PBS) were added to the vials as shown in Table 5, and mixed using a blunt needle to form a paste. The paste was gently overlayed with an additional 2 ml of PBS.

TABLE 5
Compositions for protein release experiment.

| | | | bovine | | |
|---|---|---|---|---|---|
| | | | lactoferrin (10 PBS | PBS | |
| Vial | Bone dust | HDM | mg/ml) | overlay | |
| 1-3 | 100 mg | | | 500 μl | 2 ml |
| 4-6 | 100 mg | 100 mg | | 500 μl | 2 ml |
| 7-9 | 100 mg | 100 mg | 200 μl | 300 μl | 2 ml |
| 10-12 | | 100 mg | | 500 pl | 2 ml |

The vials were incubated at room temperature without agitation, and 100 μl samples of the top aqueous phase were taken at 24,48, and 72 hours, replacing with 100 μl PBS each time. The protein content of the samples was then measured.

### 5.2 Results

Immediately after preparation, the BD only vials (1-3) showed PBS with a clear, light “bloody” colour. The BD+HDM vials (4-6) and BD+HDM+bLF vials (7-9) showed a darker red BD/HDM gel phase at the bottom, with BD and a pinky/white middle gel phase, and a clear aqueous phase at the top. The HDM only vials (10-12) had a large white HDM gel phase taking up ˜⅔ of the volume, and an aqueous phase on top.

At 24 hours, the BD only vials showed a more “bloody” colour; the BD+HDM and BD+HDM+bLF middle phase appeared pinker and broader, and there was a clear aqueous phase at the top. The HDM only vials showed a slightly wider white HDM gel phase and a slightly smaller aqueous phase.

At 48 hours, the BD only vials showed PBS with a darker, reddish colour; and the BD+HDM, BD+HDM+bLF, and BDM only vials were similar to the 24-hour timepoint.

At 72 hours, the BD only vials were similar to the 48-hour timepoint; the BD+HDM and BD+HDM+bLF vials lacked a clear aqueous phase at the top. The HDM only vials still showed a white HDM gel phase with a clear aqueous phase on top.

The concentration of protein present in the samples of aqueous phase at each timepoint is shown in FIG. 5. The BD only samples showed a similar high protein concentration at each timepoint. The BD+HDM and BD+HDM+bLF samples showed a slowly increasing protein concentration over time, with the BD+HDM+bLF samples having released more protein at each timepoint than the BD+HDM samples.

### 5.3 Conclusions

This example shows that the use of biocompatible modified starch particles to form a hydrogel matrix incorporating bone dust can slow the release of proteins from the matrix, providing release over an extended time period compared to bone dust alone. This example also suggests that other bioactive molecules, such as lactoferrin, that are incorporated into the hydrogel matrix can also be released over time.

### REFERENCES

Gao, Ryan, Matthew Street, Mei L. Tay, Karen E. Callon, Dorit Naot, Alistair Lock, Jacob T. Munro, Jillian Cornish, John Ferguson, and David Musson. 2018. ‘Human Spinal Bone Dust as a Potential Local Autograft: In Vitro Potent Anabolic Effect on Human Osteoblasts’. Spine 43 (4): E193-99. https://doi.org/10.1097/BRS.0000000000002331.

Hernigou, Philippe. 2014. ‘Bone Transplantation and Tissue Engineering, Part I. Mythology, Miracles and Fantasy: From Chimera to the Miracle of the Black Leg of Saints Cosmas and Damian and the Cock of John Hunter’. International Orthopaedics 38 (12): 2631-38. https://doi.org/10.1007/s00264-014-2511-y.

Meyers, Carolyn, Jeffrey Lisiecki, Sarah Miller, Adam Levin, Laura Fayad, Catherine Ding, Takashi Sono, Edward McCarthy, Benjamin Levi, and Aaron W. James. 2019. ‘Heterotopic Ossification: A Comprehensive Review’. JBMR Plus 3 (4): e10172. https://doi.org/10.1002/jbm4.10172.

Older, M.W.J. 1992. ‘Introduction: History and Early Research on Bone Transplantation’. In Bone Implant Grafting, 14-18. London: Springer Verlag.

Schmidt, Andrew H. 2021. ‘Autologous Bone Graft: Is It Still the Gold Standard?’ Injury 52 (June): S18-22. https://doi.org/10.1016/j.injury.2021.01.043.

Street, Matthew, Ryan Gao, Waldron Martis, Jacob Munro, David Musson, Jillian Cornish, and John Ferguson. 2017. ‘The Efficacy of Local Autologous Bone Dust: A Systematic Review’. Spine Deformity 5 (4): 231-37. https://doi.org/10.1016/j.jspd.2017.02.003.

Younger, Edward M., and Michael W. Chapman. 1989. ‘Morbidity at Bone Graft Donor Sites’: Journal of Orthopaedic Trauma 3 (3): 192-95. https://doi.org/10.1097/00005131-198909000-00002.

### INDUSTRIAL APPLICABILITY

This disclosure relates to methods of delivering a biological material to a wounded tissue of a patient comprising the application of a composition comprising biocompatible modified starch particles, wherein the starch particles are modified by carboxylation or hydroxylation of glucose units in the starch.

What we claim is:

1. A method of delivering a biological material to a wounded tissue of a patient, the method comprising:
applying a mixture to the wounded tissue of the patient, the mixture having been obtained by combining to form a mixture,
a. a composition comprising biocompatible modified starch particles, wherein the starch particles are modified by carboxylation or hydroxylation of glucose units in the starch, and
b. the biological material.

2. The method of claim 1, wherein the biological material comprises a biological fluid.

3. The method of claim 2, wherein the biological fluid comprises blood, blood plasma, and/or lymph.

4. The method of claim 1, wherein the biological material comprises one or more tissue fragments.

5. The method of claim 4, wherein the one or more tissue fragments comprise autogenous tissue fragments from the patient.

6. The method of claim 4, wherein the one or more tissue fragments comprise allogenic tissue fragments from a donor.

7. The method of claim 4, wherein the tissue fragments comprise bone dust or cartilage.

8. The method of claim 1, wherein the mixture comprises growth factors, cytokines, anabolic factors, osteoinductive factors, and/or viable cells.

9. The method of claim 1, wherein the mixture has a viscosity at 25° C. of at least 50 mPa·s,

10. The method of claim 9, wherein the mixture has a viscosity at 25° C. of from 30,000 to 10⁶ mPa·s.

11. The method of claim 1, wherein the biological material is combined with the biocompatible modified starch particles in a ratio of from about 5:1 to about 20:1.

12. The method of claim 1, wherein the composition comprising biocompatible modified starch particles is in the form of:
a. a powder; or
b. a gel, putty, or paste.

13. The method of claim 1, wherein the method comprises delivering a biological material in lumbar fusion surgery, maxillofacial surgery, oral surgery, dental surgery, or cranial surgery.

14. The method of claim 13, wherein the biological material is harvested from material removed during the surgery.

15. The method of claim 1, wherein the combining comprises combining:
a. a composition comprising biocompatible modified starch particles, wherein the starch particles are modified by carboxylation or hydroxylation of glucose units in the starch,
b. the biological material, and
c. a liquid.

16. The method of claim 15, wherein the ratio of biocompatible modified starch particles to liquid is from 1:1 to 1:30.

17. The method of claim 1, wherein the composition comprises one or more bioactive agents selected from osteogenic, anti-inflammatory, antimicrobial, antibacterial, and/or antibiotic agents.

18. The method of claim 17, wherein the one or more bioactive agents comprises lactoferrin.

19. The method of claim 1, wherein the combining comprises combining:
a. a composition comprising biocompatible modified starch particles, wherein the starch particles are modified by carboxylation or hydroxylation of glucose units in the starch,
b. the biological material, and
c. one or more bioactive agents selected from osteogenic, anti-inflammatory, antimicrobial, antibacterial, and/or antibiotic agents.

20. The method of claim 1, wherein the biocompatible modified starch particles are:
a. degradable by amylase;
b. carboxymethyl starch particles;
c. cross-linked;
d. pre-gelatinised modified starch particles;
e. non-porous;
f. substantially devoid of a microporous surface; or
g. any combination of any two or more of (a) to (f).

21. A composition comprising:
a. biocompatible modified starch particles;
b. optionally a liquid; and
c. a biological material;
wherein the composition is malleable and cohesive and/or
wherein the weight ratio of biocompatible modified starch particles to liquid and/or biological material is from 1:1 to 1:50.

22. The composition of claim 21, wherein the liquid comprises saline.

23. The composition of claim 21, wherein the composition has a viscosity at 25° C. of at least 50 mPa·s.

24. A method for preparing a mixture for delivering a biological material to a wounded tissue of a patient, comprising combining:
a. a composition comprising biocompatible modified starch particles, wherein the starch particles are modified by carboxylation or hydroxylation of glucose units in the starch,
b. a biological material, and
c. optionally saline,
thus obtaining the mixture, wherein the mixture is malleable and cohesive.
